(19) United States
(12) Patent Application Publication
(10) Pub. No.: US 2007/0287944 A1
(43) Pub. Date: Dec. 13, 2007

(54) Thoracic Compression Device

(76) Inventors: Isabel Ruiz Escalera, Barcelona (ES); Daniel Claramunt Artigas, Barcelona (ES)

Correspondence Address: BANNER & WITCOFF, LTD., 1100 13th STREET, N.W., SUITE 1200, WASHINGTON, DC, 20005-4051, US

(21) Appl. No.: 11/737,435

(22) Filed: Apr. 19, 2007

(30) Foreign Application Priority Data
Apr. 27, 2006 (ES) 200601080

(51) Int. Cl. A61F 5/00

(52) U.S. Cl. 602/20; 602/5

(57) ABSTRACT

The present invention refers to a thoracic compression device. The device is designed for use in the field of medicine in cases where compression and holding of the thoracic zone is required, for example following breast surgery or in the case of rib injuries or any other situation requiring compression and hold. The device is characterised in that it comprises a main textile body which covers at least the hemithorax and which is provided with the means for regulating the compression exerted and with means for holding at least one of the patient's shoulders.

### OBJECT OF THE INVENTION

The present invention refers to a thoracic compression device. The device is designed for use in the field of medicine in cases where compression and holding of the thoracic zone is required, for example following breast surgery or in the case of rib injuries or any other situation requiring compression and hold.

The device is characterised in that it comprises a main textile body which covers at least the hemithorax and which is provided with the means for regulating the compression exerted and with means for holding at least one of the patient's shoulders.

### BACKGROUND TO THE INVENTION

It is well known that following surgical intervention of the breast or in the case of rib injuries it is necessary to compress and hold the damaged zone in order to facilitate recovery.

In breast operations this compression prevents hemorrhage, haematomas, seromas, etc. whereas in rib injuries it is used to provide support in the injured zone and to alleviate pain.

At present the function of compression is carried out by bandaging the patent with bands or gauze which may be self adhesive. This system has the disadvantage that when removing the bandage the patient is subjected to the pain produced by detaching the adhesive

The invention described here overcomes these disadvantages by means of a device which provides compression and hold which may be regulated both in terms of the area requiring the greatest compression and in the case in which greater pressure is required, for example in the hours immediately following surgical intervention. Another advantage of the present device is that it may compress a single haemothorax so that the undamaged rib would remain compressed, in this way, ensuring that the patient does not suffer any difficulty with breathing.

The invention described also has the advantage of the fact that by using a textile fabric which does not adhere to the patient's skin it avoids abrasions, skin rashes or blisters.

### DESCRIPTION OF THE INVENTION

The present invention refers to a thoracic compression device. The device is designed for use in the field of medicine in cases where compression and holding of the thoracic zone is required, for example following breast surgery or in the case of rib injuries or any other situation requiring compression and holding.

The device is characterised in that it comprises a main textile body which covers at least the hemithorax and which is provided with the means for regulating the compression exerted and means for holding at least one shoulder.

The means for regulating the compression exerted consists of the extension of the main body in bands which surround at least the damaged hemithorax until it links once more with the main body.

The means for holding the main body to the shoulder consists of a band with an adjustable height which starts from the main body and surrounds the shoulder.

### DESCRIPTION OF THE DRAWINGS

The present descriptive report is supplemented by a series of drawings illustrative of a preferred embodiment but not however restricting the invention in any way.

FIG. 1 shows a diagram of the front part of the compression device.

FIG. 2 shows a diagram of the back part of the compression device.

FIG. 3 is a representation in extended section of the main body and the bands.

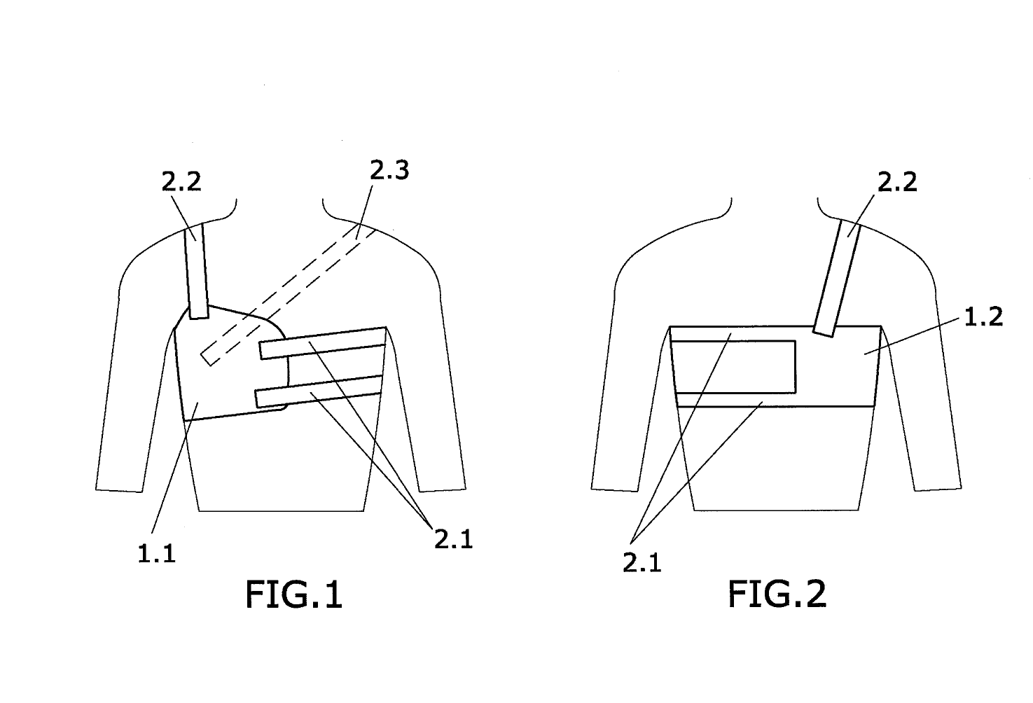

### PREFERRED EMBODIMENT OF THE INVENTION

The present invention refers to a thoracic compression device. The device described is applicable in the field of medicine, in those cases in which the thoracic area needs to be compressed and supported.

The device described is characterised in that it comprises a main textile body (1) which covers at least the hemithorax and which is provided with the means for adjusting the compression exerted and with the means for holding at least one of the patient's shoulders.

In the preferred embodiment the means for adjusting horizontal compression consists of the extension of the main body (1) into bands (2.1) which surround at least the undamaged hemithorax until it links again with the main body (1). The bands (2.1) start from the back area of the main body (1) and are joined thereto (1) in the front area.

The means for holding the main body (1) to the patient's shoulder also consist of a band (2.2) with an adjustable height which connects with the front part of the main body (1) with the back part passing over the shoulder.

The connection between the bands (2.1, 2.2) and the main body (1) is achieved through a physical adherence system or based on chemical products.

In the preferred embodiment the adhesive system consists of the combination of Velcro in the bands (2.1, 2.2) and a textile fabric of the main body (1) appropriate for adherence thereto. Thus, in a simple system based on the position in which the bands are stuck to the main body, the compression exerted by the device can be varied.

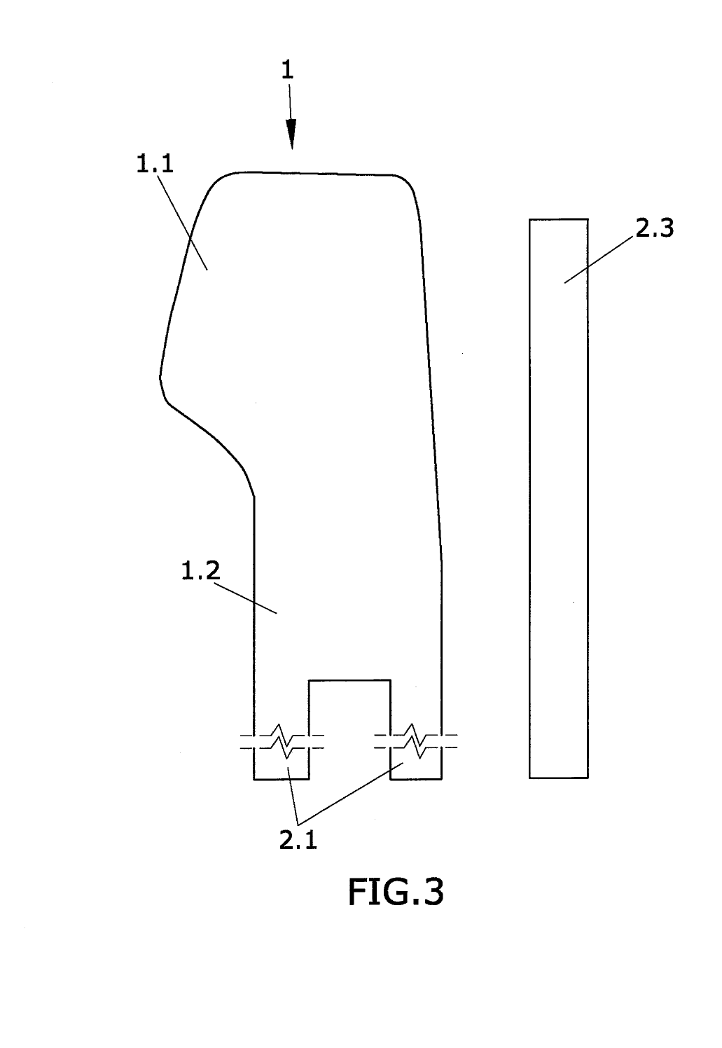

The main body (1) of the preferred embodiment is provided with a front zone (1.1) which is wider than the back zone (1.2) as may be seen in FIG. 3.

The device is also provided with bands of extra compression (2.3) with an adhesive surface at both ends which provide greater compression in those zones or at those times when this is required.

The main textile from which both the main body (1) and the bands (2.1, 2.2, 2.3) are made may be quilted for the patient's extra comfort

The essential nature of this invention is not altered in any way by variations in materials, form, shape and arrangement of the component elements which are described in a manner which is in no way restrictive but which is sufficient to for an expert to proceed to its reproduction.

1. Thoracic compression device, characterised in that it consists of a main textile body (1) which covers at least the hemithorax and which is provided with the means for adjusting the compression exerted and the means for holding at least one shoulder.

2. Thoracic compression device according to claim 1, characterised in that the means for adjusting compression consists of the extension of the main body (1) into bands (2.1) which surround at least the healthy hemithorax until it links again with the main body (1).

3. Thoracic compression device according to claim 1 characterised in that the means for holding it to the shoulder consist of a band (2.2) with an adjustable length.

4. Thoracic compression device according to previous claims (2.1, 2.2) and main body (1) is carried out through an adhesive medium.

5. Thoracic compression device according to claim 4, characterised in that the band (2.2) which surrounds the shoulder is adhesive at both ends.

6. Thoracic compression device according to claim 4 characterised in that the adhesive medium consists of a combination of velcro in the bands (2.1, 2.2) and a textile fabric of the main body (1) appropriate for adherence thereto.

7. Thoracic compression device according to claim 1, characterised in that the main body (1) is provided with a front area (1.1) with greater width than the back area (1.2).

8. Thoracic compression device according to claim 1, characterised in that it is provided with adhesive bands (2.3) which reinforce compression.

9. Thoracic compression device according to claim 1, characterised in that the fabric of the main body (1) may be quilted.
